(19) United States
(12) Patent Application Publication
(10) Pub. No.: US 2023/0220438 A1
(43) Pub. Date: Jul. 13, 2023

(54) COMPOSITIONS AND METHODS FOR AUTO-INDUCIBLE CELLULAR LYSIS AND NUCLEOTIDE HYDROLYSIS

(71) Applicant: Duke University, Durham, NC (US)

(72) Inventors: Michael David Lynch, Durham, NC (US); Romel Menacho Melgar, Durham, NC (US); Eirik Adim Moreb, Durham, NC (US); John P. Efromson, Durham, NC (US)

(73) Assignee: Duke University, Durham, NC (US)

(21) Appl. No.: 17/758,477

(22) PCT Filed: Jan. 8, 2021
(86) PCT No.: PCT/US21/12605

§ 371 Date: Jul. 7, 2022

Related U.S. Application Data
(60) Provisional application No. 62/958,806, filed on Jan. 9, 2020.

(51) Int. Cl. C12P 21/02; C12N 9/36; C12N 9/16; C12N 1/06

(52) U.S. Cl. CPC C12P 21/02; C12N 9/2462; C12N 9/16; C12Y 301/30002; C12N 1/06

(57) ABSTRACT

An improved strain of E. coli for autoinduction of protein expression but also of autolytic enzymes thereby enabling combined autolysis and auto DNA/RNA hydrolysis. This combination of these two mechanisms improves cellular lysis and DNA removal and expounds the benefits of two stage production of a protein product. This system enables greater than 95% lysis and hydrolysis due to tightly controlled expression the genes. The autolytic genes may encode a lysozyme and a benzonase.

### CROSS-REFERENCE TO RELATED APPLICATIONS

This application claims priority to U.S. Provisional Patent Application No. 62/958,806, filed Jan. 9, 2020, which is incorporated by reference herein in its entirety.

### STATEMENT AS TO FEDERALLY SPONSORED RESEARCH

This invention was made with Government support under Federal Grant nos.: HR0011-14-C-0075 awarded by the Defense Advanced Research Projects Agency (DARPA); YIP #12043956 awarded by the Office of Naval Research; and EE0007563 awarded by the Department of Energy (DOE). The Federal Government has certain rights to this invention.

### REFERENCE TO A SEQUENCE LISTING

The instant application contains a Sequence Listing which has been filed electronically in ASCII format as 47381-44_ST25.txt created on Jan. 3, 2021 and is 1954 bytes in size and is hereby incorporated by reference in its entirety.

### BACKGROUND

E. coli is a mainstay for routine expression of recombinant proteins. Recent estimates indicate that over 70% of laboratory studies, reliant on heterologous proteins, utilize E. coli. This microbe is commonly used in workflows ranging from high throughput screens, to routine shake flask expression and larger scale fermentations. In addition, E. coli is also used for the manufacturing of proteins at large scale, including the production of over 30% of protein-based drugs. A key challenge to the use of E. coli as well as other expression systems where proteins are not secreted, is the recovery of protein from the cell, which routinely requires cell lysis. Common laboratory methods for lysis include: chemical (base or detergents), biochemical (lysozyme) as well as mechanical methods (cell disruptors, french press or sonication), which can not only be tedious and time consuming but yield inconsistent results. Certain proteins may not tolerate the use of chemical lysis buffers and mechanical methods can lead to incomplete lysis and release of target proteins. In addition, mechanical methods are not amenable to certain workflows such as high throughput screening. At larger scales, homogenizers are often used to enable more consistent cell lysis, but these units are both costly and add additional steps to commercial processes. Significant efforts have been made in developing methods for rapid, consistent cell lysis, including engineering of E. coli strains for autolysis, usually upon induction of one more proteins with lytic activity including: lysozyme, D-amino acid oxidase, muramidase and bacterial phage lysis proteins, which are induced in parallel with proteins of interest and activated after cells are harvested.

Key remaining challenges with many of these approaches include additional process steps, incomplete lysis or additional induction procedures or vectors. In addition, previous efforts have been focused on cell wall lysis and protein release without consideration of lysate clarification to remove oligonucleotide contamination as well as reduce lysate viscosity. In commercial production after cell lysis, nucleases such as benzonase or alternatives are often used to remove nucleotide contaminants and reduce lysate viscosity to enable easier follow on purification. Benzonase is a small nonspecific extracellular nuclease from Serratia marcescens, that is routinely used to hydrolyze contaminating nucleotides during protein purification and has activity with both double stranded and single stranded DNA as well as RNA. An engineered strain of E. coli has been reported with periplasmic expression of a nuclease which auto-hydrolyzes host nucleic acids upon cell lysis, but autolysis and autohydrolysis have yet to be combined. Hence, there remains a need to refine methods for enhancing recombinant protein production.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

In some aspects, an engineered E. coli microorganism characterized by controlled autoinduction of cellular autolysis or DNA/RNA auto hydrolysis, is provided. The genes may encode a periplasmic lysozyme and/or a cytoplasmic nuclease. The genes are preferably operatively linked to a promoter that induces gene expression upon a trigger. In some aspects, the depletion of a nutrient from media containing the microorganism may be the triggering event. In some cases the nutrient that is depleted from microsomal media is phosphate. In another aspect, the inducible promotor and the genes encoding a periplasmic lysozyme or the genes encoding a cytoplasmic nuclease microorganism are integrated as an operon in the chromosome of the microorganism.

In certain aspects, the periplasmic lysozyme is a lambda phage lysozyme, or the periplasmic lysozyme is the Lambda R gene.

In certain aspects, the cytoplasmic nuclease is a benzonase, or the Serratia marcescens nucA gene.

In some aspects, the engineered E. coli microorganism further includes a pathway for heterologous protein production by the microorganism. In this case, genes encoding enzymes essential for heterologous protein production are also operatively linked to a promoter that induces gene expression upon depletion of a nutrient from media containing the microorganism.

In some aspects, a method of cellular lysis and protein recovery is provided. Firstly, an engineered E. coli microorganism comprising tightly controlled autolytic enzymes is provided. As a second step, the microorganism is grown in a nutrient limited media. Thirdly, a microorganism stationary phase is induced upon nutrient depletion. The next step involves disrupting cell wall or membrane integrity and finally collecting protein product.

In another aspect, the method provides for production of a heterologous product. In this case, the engineered E. coli microorganism further comprised genes encoding enzymes essential for heterologous protein production operatively linked to a promoter that induces gene expression upon depletion of a nutrient from media in the growth phase.

In another aspect, the methods use engineered E. coli microorganism including both genes encoding a periplasmic lysozyme and genes encoding a cytoplasmic nuclease as the autolysis genes.

In another aspect, the step of disrupting cell wall or membrane integrity comprises at least one freeze thaw cycle, agitation, detergent addition or a combination of techniquest

Other methods, features and/or advantages is, or will become, apparent upon examination of the following figures and detailed description. It is intended that all such additional methods, features, and advantages be included within this description and are protected by the accompanying claims.

### BRIEF DESCRIPTION OF DRAWINGS

The novel features of the invention are set forth with particularity in the claims. A better understanding of the features and advantages of the present invention will be obtained by reference to the following detailed description that sets forth illustrative aspects, in which the principles of the invention are used, and the accompanying drawings of which:

FIG. 1A-D are graphs and schematics showing the overview of 2-stage autolysis/hydrolysis in accordance with one aspect of the present disclosure.

FIG. 2A-C are are graphs showing the growth and autoinduction of the autolysis/hydrolysis strain (DLF_R004) as compared to a non-autolytic controls (DLF_R002 and DLF_R003) in accordance with one aspect of the present disclosure.

FIG. 3A-B are graphs showing autolysis and protein release of strains DLF_R004 (autolysis/hydrolysis strain) and DLF_R003 (control) in accordance with one aspect of the present disclosure.

FIG. 4 is a graph showing the impact of Triton™ level of lysis and protein release in accordance with one aspect of the present invention.

FIG. 5 is a graph demonstrating the impact of freeze thaw cycles without Triton™ additions on protein release.

FIG. 6A-B are graphs demonstrating DNA hydrolysis in DLF_R004-pHCKan-GFPuv. A: Agarose gel electrophoresis of heat denatured lysates with EDTA present from the beginning of lysis. B: Agarose gel electrophoresis of heat denatured lysates with active benzonase.

FIG. 7A-C are images of electrophoresis gels and graphs showing the autohydrolysis of RNA/DNA of strain DLF_R004 and time course of autolysis and GFPuv release un under autohydrolysis conditions using strain DLF_R004 bearing plasmid pHCKan-yibDp-GFPuv in accordance with one aspect of the present disclosure.

FIG. 8 is a flow diagram representing an exemplary autoinducible lysis and hydrolysis shake flask protocol.

FIG. 9 is a pictorial representation of a sample of cleared lysate.

FIG. 10 is a graph showing autolysis and protein release in 96 well microtiter plates in accordance with one aspect of the invention.

FIG. 11 is a graph showing the stability of uninduced strain DLF_R004 (autolysis/hydrolysis strain) in accordance with one aspect of the present disclosure.

FIG. 12 is a table describing the sequences of DNA and oligonucleotides.

### DETAILED DESCRIPTION

### 1. Definitions

Unless otherwise defined, all technical and scientific terms used herein have the same meaning as commonly understood by one of ordinary skill in the art to which this invention pertains. In case of conflict, the present specification, including definitions, will control.

Unless otherwise specified, “a,” “an,” “the,” “one or more of,” and “at least one” are used interchangeably. The singular forms “a”, “an,” and “the” are inclusive of their plural forms.

“About” is used to provide flexibility to a numerical range endpoint by providing that a given value may be “slightly above” or “slightly below” the endpoint without affecting the desired result The recitations of numerical ranges by endpoints include all numbers subsumed within that range (e.g., 0.5 to 5 includes 1, 1.5, 2, 2.75, 3, 3.80, 4, 5, etc.).

The terms “comprising” and “including” are intended to be equivalent and open-ended. The phrase “consisting essentially of” means that the composition or method may include additional ingredients and/or steps, but only if the additional ingredients and/or steps do not materially alter the basic and novel characteristics of the claimed composition or method. The phrase “selected from the group consisting of” is meant to include mixtures of the listed group. As used herein, “and/or” refers to and encompasses any and all possible combinations of one or more of the associated listed items, as well as the lack of combinations where interpreted in the alternative (“or”).

Moreover, the present disclosure also contemplates that in some aspects, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

The term “heterologous DNA,” “heterologous nucleic acid sequence,” and the like as used herein refers to a nucleic acid sequence wherein at least one of the following is true: (a) the sequence of nucleic acids is foreign to (i.e., not naturally found in) a given host microorganism; (b) the sequence may be naturally found in a given host microorganism, but in an unnatural (e.g., greater than expected) amount; or (c) the sequence of nucleic acids comprises two or more subsequences that are not found in the same relationship to each other in nature. For example, regarding instance (c), a heterologous nucleic acid sequence that is recombinantly produced will have two or more sequences from unrelated genes arranged to make a new functional nucleic acid, such as a nonnative promoter driving gene expression. The term “heterologous” is intended to include the term “exogenous” as the latter term is generally used in the art. With reference to the host microorganism's genome prior to the introduction of a heterologous nucleic acid sequence, the nucleic acid sequence that codes for the enzyme is heterologous (whether or not the heterologous nucleic acid sequence is introduced into that genome). As used herein, chromosomal and native and endogenous refer to genetic material of the host microorganism.

As used herein, the term “gene disruption,” or grammatical equivalents thereof (and including “to disrupt enzymatic function,” “disruption of enzymatic function,” and the like), is intended to mean a genetic modification to a microorganism that renders the encoded gene product as having a reduced polypeptide activity compared with polypeptide activity in or from a microorganism cell not so modified. The genetic modification can be, for example, deletion of the entire gene, deletion or other modification of a regulatory sequence required for transcription or translation, deletion of a portion of the gene which results in a truncated gene product (e.g., enzyme) or by any of various mutation strategies that reduces activity (including to no detectable activity level) the encoded gene product. A disruption may broadly include a deletion of all or part of the nucleic acid sequence encoding the enzyme, and also includes, but is not limited to other types of genetic modifications, e.g., introduction of stop codons, frame shift mutations, introduction or removal of portions of the gene, and introduction of a degradation signal, those genetic modifications affecting mRNA transcription levels and/or stability, and altering the promoter or repressor upstream of the gene encoding the enzyme.

When the genetic modification of a gene product, i.e., an enzyme, is referred to herein, including the claims, it is understood that the genetic modification is of a nucleic acid sequence, such as or including the gene, that normally encodes the stated gene product, i.e., the enzyme.

Species and other phylogenic identifications are according to the classification known to a person skilled in the art of microbiology.

Enzymes are listed here within, with reference to a UniProt identification number, which would be well known to one skilled in the art. The UniProt database can be accessed at http://www.UniProt.org/. When the genetic modification of a gene product, i.e., an enzyme, is referred to herein, including the claims, it is understood that the genetic modification is of a nucleic acid sequence, such as or including the gene, that normally encodes the stated gene product, i.e., the enzyme.

Where methods and steps described herein indicate certain events occurring in certain order, those of ordinary skill in the art will recognize that the ordering of certain steps may be modified and that such modifications are in accordance with the variations of the invention. Additionally, certain steps may be performed concurrently in a parallel process when possible, as well as performed sequentially.

The meaning of abbreviations is as follows: “C” means Celsius or degrees Celsius, as is clear from its usage, DCW means dry cell weight, “s” means second(s), “min” means minute(s), “h,” “hr,” or “hrs” means hour(s), “psi” means pounds per square inch, “nm” means nanometers, “d” means day(s), “4” or “uL” or “ul” means microliter(s), “mL” means milliliter(s), “L” means liter(s), “mm” means millimeter(s), “nm” means nanometers, “mM” means millimolar, “μM” or “uM” means micromolar, “M” means molar, “mmol” means millimole(s), “μmol” or “uMol” means micromole(s)”, “g” means gram(s), “μg” or “ug” means microgram(s) and “ng” means nanogram(s), “PCR” means polymerase chain reaction, “OD” means optical density, “OD600” means the optical density measured at a photon wavelength of 600 nm, “kDa” means kilodaltons, “g” means the gravitation constant, “bp” means base pair(s), “kbp” means kilobase pair(s), “% w/v” means weight/volume percent, “% v/v” means volume/volume percent, “IPTG” means isopropyl-μ-D-thiogalactopyranoiside, “aTc” means anhydrotetracycline, “RBS” means ribosome binding site, “rpm” means revolutions per minute, “HPLC” means high performance liquid chromatography, and “GC” means gas chromatography.

### 2. Carbon Sources

Growth media, which is used in the present disclosure with recombinant microorganisms (e.g., E coli) must contain suitable carbon sources or substrates for both growth and production stages. Suitable substrates may include, but are not limited to glucose, sucrose, xylose, mannose, arabinose, oils, carbon dioxide, carbon monoxide, methane, methanol, formaldehyde and glycerol. It is contemplated that all of the above-mentioned carbon substrates and mixtures thereof are suitable in the present invention as a carbon source(s).

### 3. Microorganisms

Features as described and claimed herein may be provided in a microorganism that comprises one or more natural, introduced, or enhanced product high-production pathways. Thus, in some aspects the microorganism(s) comprise an endogenous product production pathway (which may, in some such aspects, be enhanced), whereas in other aspects the microorganism does not comprise an endogenous product production pathway. In some aspects, the microorganism comprises E. coli. Though the methods and genes described herein are applicable to any microorganism species including: for example Acinetobacter calcoaceticus, Bacillus subtilis, Chlorobium limicola, Citrobacter braakii, Clostridium acetobutylicum, Clostridium aminobutyricum, Clostridium kluyveri, Cornyebacterium glutamicum, Cupriavidus metallidurans, Cupriavidus necator, Desulfovibriofructosovorans, Escherichia coli strain BW25113, Escherichia coli strain BWapldfis, Halobacterium salinarum, Lactobacillus delbrueckii, Metallosphaera sedula, Methylococcus capsulatus, Methylococcus thermophilus IMV 2, Methylosinus tsporium, Pichia pastoris (Komagataella pastoris, Propionibacterium freudenreichii subsp. Shermanii, Pseudomonas putida, Saccharomyces cerevisiae, Streptococcus mutans, or Yarrowia lipolytica.

### 4. Media and Culture Conditions

In addition to an appropriate carbon source, such as selected from one of the herein disclosed types, growth media must contain suitable minerals, salts, cofactors, buffers and other components, known to those skilled in the art, suitable for the growth of the cultures and promotion of protein product production under the present disclosure.

Typically, cells are grown at a temperature in the range of about 25° C. to about 40° C. in an appropriate medium, as well as up to 70° C. for thermophilic microorganisms. Suitable growth media are well characterized and known in the art. Suitable pH ranges for the bio-production are between pH 2.0 to pH 10.0, where pH 6.0 to pH 8.0 is a typical pH range for the initial condition. However, the actual culture conditions for a particular aspect are not meant to be limited by these pH ranges. Growth of the microorganisms may be performed under aerobic, microaerobic or anaerobic conditions with or without agitation.

In addition to an appropriate carbon source, such as selected from one of the herein-disclosed types, growth media must contain suitable minerals, salts, cofactors, buffers and other components, known to those skilled in the art, suitable for the growth of the cultures and promotion of protein production under the present disclosure.

### 5. Triggers for Inducing a Stationary Phase within an Engineered Microorganism

Means for inducing a stationary phrase within a microorganism may include but are not limited to artificial chemical inducers including: tetracycline, anhydrotetracycline, lactose, IPTG (isopropyl-beta-D-1-thiogalactopyranoside), arabinose, raffinose, tryptophan and numerous others. Systems linking the use of these well known inducers to the control of gene expression can be integrated into genetically modified microbial systems to control the transition between growth and protein product production phases. Additionally, the transition between growth and a stationary phase may occur via depletion of one or more limiting nutrients that are consumed during growth. Limiting nutrients can include but are not limited to: phosphate, inorganic phosphate, nitrogen, sulfur and magnesium.

### 6. Overview of Invention Aspects

Accordingly, one aspect of the present disclosure provides an engineered E. coli comprising, consisting of, or consisting essentially of one or more genes encoding periplasmic lysozyme and/or cytoplasmic nuclease, wherein the genes encoding periplasmic lysozyme and/or cytoplasmic nuclease are expressed under the control of at least one promoter induced under phosphate limiting conditions, wherein induction of expression of said promoter is initiated upon phosphate depletion.

In some aspects, an engineered E. coli microorganism characterized by controlled autoinduction of cellular autolysis or DNA/RNA auto hydrolysis, is provided. The genes may encode a periplasmic lysozyme or a cytoplasmic nuclease. The genes are preferably operatively linked to a promoter that induces gene expression upon a trigger. In some aspects, the depletion of a nutrient from media containing the microorganism may be the triggering event. In some cases, the nutrient that is depleted from microsomal media is phosphate. Operatively linked merely indicates the genes and promotor are in relationship with each other. This phrase also applies if an addition of additional sequences or more than one promotor is present. More than one genes may be linked to the same promotor or group of promotors or the genes may form an operon in which more than one gene is controlled by the same promotor.

In one aspect, the engineered E. coli microorganism may include both genes encoding a periplasmic lysozyme and genes encoding a cytoplasmic nuclease that together are considered autolytic enzymes. Similarly, the autolytic enzyme group may include more than only periplasmic lysozyme gene and/or more than one cytoplasmic nuclease. The autolytic enzyme may group encompasses any lysozyme and any nuclease.

In another aspect, the engineered E. coli microorganism the inducible promotor and the genes encoding a periplasmic lysozyme or the genes encoding a cytoplasmic nuclease microorganism are integrated as an operon in the chromosome of the microorganism.

In another aspect, the engineered E. coli microorganism all genes encoding a periplasmic lysozyme or all genes encoding a cytoplasmic nuclease found within the microorganism are subject to expression by inducible promoter.

In certain aspects, the periplasmic lysozyme is a lambda phage lysozyme, or the periplasmic lysozyme is the Lambda R gene.

In certain aspects, the cytoplasmic nuclease is a benzonase, or the Serratia marcescens nucA gene.

In some aspects, the engineered E. coli microorganism further includes a pathway for heterologous protein production by the microorganism. In this case, genes encoding enzymes essential for heterologous protein production are also operatively linked to a promoter that induces heterologous gene expression upon depletion of a nutrient from media containing the microorganism. In this manner both heterologous protein production and inducing of the lytic enzymes occurs synchronously when the microorganism is placed in a stationary phase. This movement from a growth phase to a stationary phase while related to inducing the heterologous gene expression and autolytic enzyme induction may also occur by a different means. That is, additional signals may induce commencement of the stationary phase in addition to gene regulation.

In some aspects, a method of cellular lysis and protein recovery is provided. Firstly, an engineered E. coli microorganism is provided. The microorganism is characterized as having autolysis genes that may include one or more genes encoding a periplasmic lysozyme or one or more genes encoding a cytoplasmic nuclease. Genes encoding the autolysis genes are operatively linked to a promoter. The promotor may induce gene expression upon depletion of a nutrient from media containing the microorganism in a growth phase. As a second step, the microorganism is grown in a nutrient limited media. Thirdly, a microorganism stationary phase is induced upon nutrient depletion. The stationary phase is characterized by: protein product expression, and induction of the expression of the autolysis genes. However, the autolysis enzymes do not induce lysis of the microorganism until cell wall or membrane integrity is disrupted. The next step involves disrupting cell wall or membrane integrity and finally collecting protein product.

In another aspect, the method provides for production of a heterologous product. In this case, the engineered E. coli microorganism further comprised genes encoding enzymes essential or necessary for heterologous protein production operatively linked to a promoter that induces gene expression upon depletion of a nutrient from media containing the microorganism, and the stationary phase is additionally characterized by induction of the expression of the heterologous genes.

In another aspect, the methods use engineered E. coli microorganism including both genes encoding a periplasmic lysozyme and genes encoding a cytoplasmic nuclease as the autolysis genes.

In another aspect, the method includes, after the step of inducing a stationary phase and prior to step of disrupting cell wall or membrane integrity, a method step of harvesting cells by centrifugation.

In another aspect, the step of disrupting cell wall or membrane integrity comprises at least one freeze thaw cycle, agitation, detergent addition or a combination thereof. In some aspects, this step includes the addition of 0.1% non-ionic detergent or enhanced nucleotide hydrolysis by including an incubation at 37° C.

### 8. Disclosed Aspects Are Non-Limiting

While various aspects of the present invention have been shown and described herein, it is emphasized that such aspects are provided by way of example only. Numerous variations, changes and substitutions may be made without departing from the invention herein in its various aspects. Specifically, and for whatever reason, for any grouping of compounds, nucleic acid sequences, polypeptides including specific proteins including functional enzymes, metabolic pathway enzymes or intermediates, elements, or other compositions, or concentrations stated or otherwise presented herein in a list, table, or other grouping unless clearly stated otherwise, it is intended that each such grouping provides the basis for and serves to identify various subset aspects, the subset aspects in their broadest scope comprising every subset of such grouping by exclusion of one or more members (or subsets) of the respective stated grouping. Moreover, when any range is described herein, unless clearly stated otherwise, that range includes all values therein and all sub-ranges therein.

Also, and more generally, in accordance with disclosures, discussions, examples and aspects herein, there may be employed conventional molecular biology, cellular biology, microbiology, and recombinant DNA techniques within the skill of the art. Such techniques are explained fully in the literature. See, e.g., Sambrook and Russell, “Molecular Cloning: A Laboratory Manual,” Third Edition 2001 (volumes 1-3), Cold Spring Harbor Laboratory Press, Cold Spring Harbor, N.Y.; Animal Cell Culture, R. I. Freshney, ed., 1986. These published resources are incorporated by reference herein.

The following published resources are incorporated by reference herein for description useful in conjunction with the invention described herein, for example, methods of industrial bio-production of chemical product(s) from sugar sources, and also industrial systems that may be used to achieve such conversion (Biochemical Engineering Fundamentals, 2^{nd} Ed. J. E. Bailey and D. F. Ollis, McGraw Hill, New York, 1986, e.g. Chapter 9, pages 533-657 for biological reactor design; Unit Operations of Chemical Engineering, 5^{th} Ed., W. L. McCabe et al., McGraw Hill, New York 1993, e.g., for process and separation technologies analyses; Equilibrium Staged Separations, P. C. Wankat, Prentice Hall, Englewood Cliffs, N.J. USA, 1988, e.g., for separation technologies teachings).

All publications, patents, and patent applications mentioned in this specification are entirely incorporated by reference.

The following Examples for Improved Two-Stage Expression and Purification Via autoinduction of both Autolysis and Auto DNA/RNA Hydrolysis Conferred by Phage Lysozyme and Benzonase are provided by way of illustration and not by way of limitation.

### EXAMPLES

### Overview

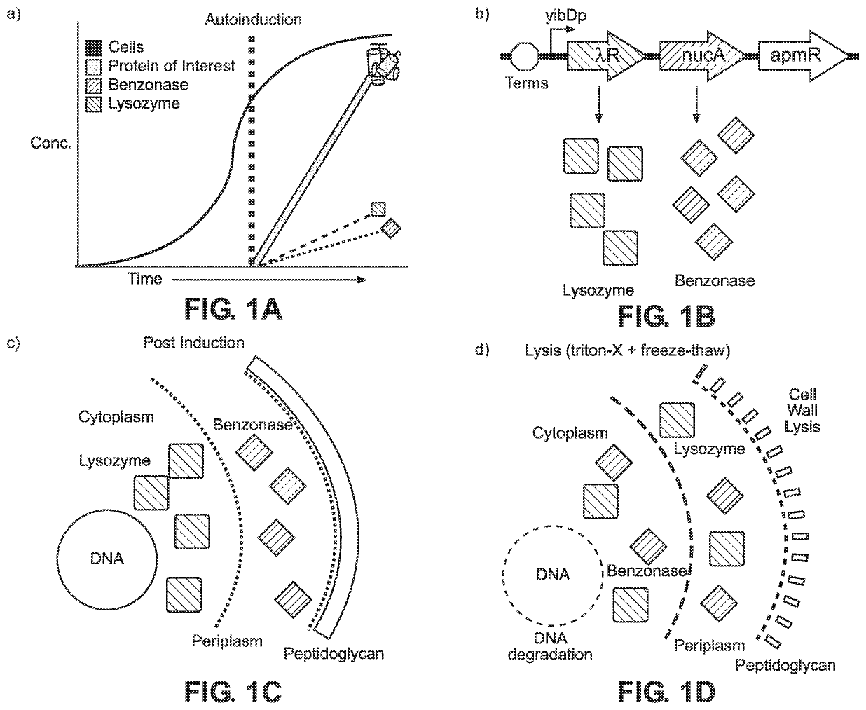

The present disclosure is based, in part, on the discovery by the inventors of improved release of recombinant proteins in E. coli, which relies on combined cellular autolysis and DNA/RNA autohydrolysis, conferred by the tightly controlled autoinduction of, for example, both phage lysozyme and benzonase. The inventors recently reported strains, plasmid and protocols for the autoinduction of protein expression in stationary phase upon batch phosphate depletion, enabling high protein titers in a very simplified protocol, with no leaky expression. To build upon this system, in this work the inventors have further engineered strains with an autolysis and autohydrolysis “module” comprising, consisting of, or consisting essentially of lambda phage lysozyme (Lambda R gene) and benzonase (encoded by the Serratia marcescens nucA gene). In some aspects, the expression of a protein of interest as well as expression of the autolysis module are induced upon phosphate depletion co incident with entry into stationary phase via phosphate regulated promoters (FIG. 1A). These two genes are integrated as an operon into the chromosome in the ompT locus (FIG. 1B), also deleting this protease, which can lead to improved protein yields tightly controlled (i.e., non-leaky) expression and autoinduction media enable a greatly simplified single step process for both high levels of expression and lysate preparation prior to further purification.

The present disclosure is based, in part, on the discovery by the inventors of improved release of recombinant proteins in E. coli, which relies on combined cellular autolysis and DNA/RNA autohydrolysis, conferred by the tightly controlled autoinduction of both phage lysozyme and benzonase. The inventors have found that autoinduction occurs in a two-stage process wherein heterologous protein expression and autolysis enzymes are induced upon entry into stationary phase by phosphate depletion. Cytoplasmic lysozyme and periplasmic benzonase are kept from inducing lysis until membrane integrity is disrupted. Post cell harvest, the addition of detergent (0.1% Triton™ X100) and a single 30 minutes freezer thaw cycle results in>90% release of protein. This cellular lysis is accompanied by complete oligonucleotide hydrolysis. The approach has been validated for shake flask cultures, high throughput cultivation in microtiter plates and larger scale stirred-tank bioreactors. This tightly controlled system enables robust growth and resistance to lysis in routine media when cells are propagated and autolysis/hydrolysis genes are only induced upon phosphate depletion.

### Example 1. Impact of Autolysis/Hydrolysis Modules on Growth and Protein Expression

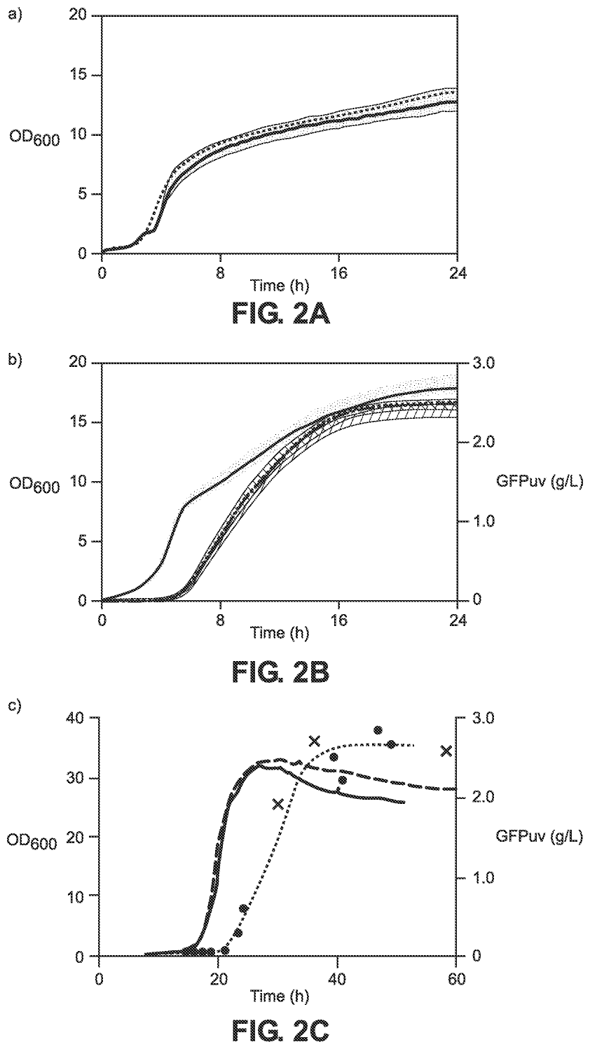

After the construction of a modified strain (DLF_R004), with integrated, phosphate regulated lysozyme and benzonase (FIG. 1B), the inventors evaluated any negative impact these modifications may have on growth and autoinduction of heterologous protein expression. Toward this aim, the inventors evaluated a autolysis/hydrolysis strain as well as its parent lacking any lysozyme or benzonase for growth and protein expression in autoinduction broth, in the M2P Labs BioLector™ (where biomass and protein expression can be monitored). Specifically, cells of either strain DLF_R004 (our autolysis/hydrolysis strain) or its parent DLF_R003, were transformed with plasmid pHCKan-yibDp-GFPuv enabling the low phosphate induction of GFP. As can be seen in FIG. 2A-B, no significant difference in growth and/or protein expression was observed when the autolysis/hydrolysis module was present.

The inventors investigated the impact of this module in instrumented bioreactors in minimal autoinduction media, where in active agitation results in increased shear stresses compared to smaller scale systems. As can be seen in FIG. 2C, no significant difference in growth and/or expression was observed indicating strain stability at least to this level of shear.

Referring specifically to FIG. 2, growth and autoinduction of the autolysis/hydrolysis strain (DLF_R004) compared to a non-autolytic control strains (DLF_R002 and DLF_R003). Black and gray line indicate biomass levels the standard error or triplicate evaluations. A: Growth of strains DLF_R004 and DLF_R003 in autoinduction broth in the M2P Labs BioLector™. DLF_R003—dashed line, DLF_R004 solid line. B: Growth and autoinduction of strain: DLF_R004 and DLF_R003 both carrying the autoinducible GFP reporter plasmid pHCKan-yibDp-GFPuv, in autoinduction broth in the M2P Labs BioLector™ DLF_R003—dashed lines, DLF_R004—solid lines. C: Growth and autoinduction in 1 L instrumented bioreactors in minimal mineral salts media. Grey lines and blue triangles, open circles and squares are three separate control experiments with strain DLF_R0042 plus pHCKan-yibDp-GFPuv data Menacho-Melgar et al, black line and green circles are results for DLF_R004 plus pHCKan-yibDp-GFPuv.

### Example 2. Autolysis

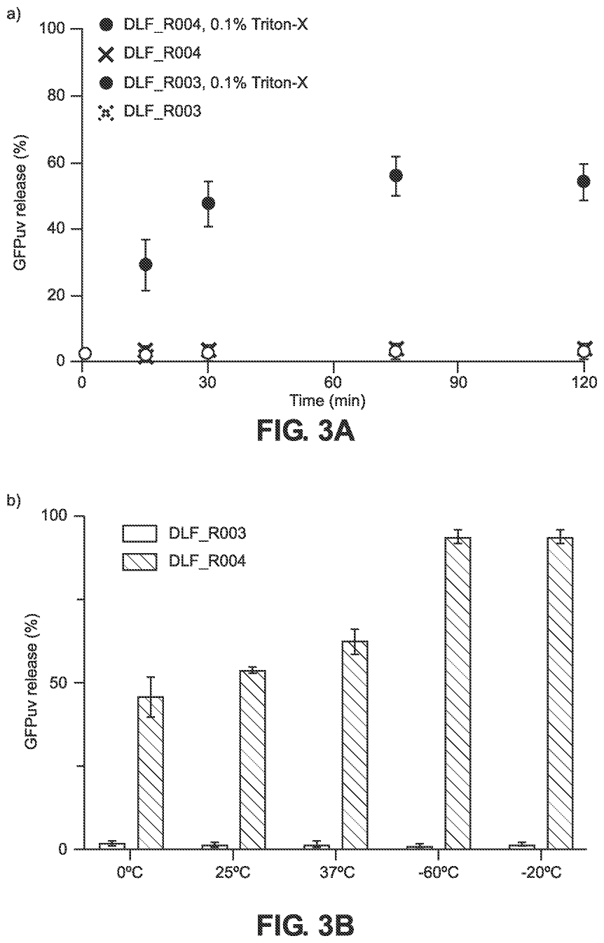

After demonstrating equivalent expression with no significant growth defects, the inventors validate the autolysis behavior of a engineered strain as shown in FIG. 3. Shake flask cultures were started in autoinduction broth (AB), and the cells were harvested by centrifugation post cell growth and GFP autoinduction. Cell pellets were washed, and Triton™-X100 was added at 0.1%. GFP release was measured over time by centrifugation and measurement of fluorescence in the supernatant (FIG. 3A).

Referring to FIG. 3 autolysis and protein release of strains DLF_R004 (autolysis/hydrolysis strain) and DLF_R003 (control) are demonstrated. A: Autolysis and GFPuv release as a function of time after the addition of Triton™-X100, cells were incubated at room temperature (25° C.). B: Autolysis and GFPuv release after the addition of 0.1% Triton-X100 and incubation for 30 minutes on ice (0° C.), room temperature (25° C.), 37° C., and a 30 minute freeze thaw at either −60° C. or −20° C.

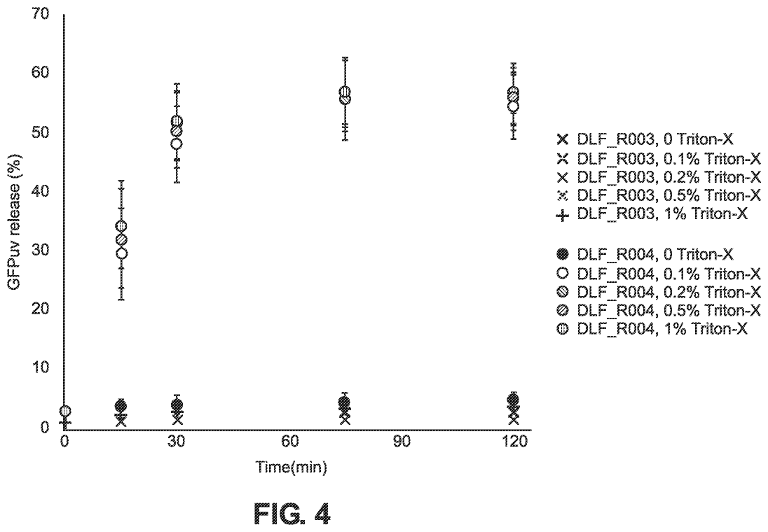

The addition of 0.1% Triton™-X100 was found to be sufficient for the release of˜55% of the total GFP in about an hour. No GFP release was observed either in the control strain or in our autolysis/hydrolysis strain without Triton™-X100 addition. Increasing Triton™-X 00 levels did not impact protein release (FIG. 4).

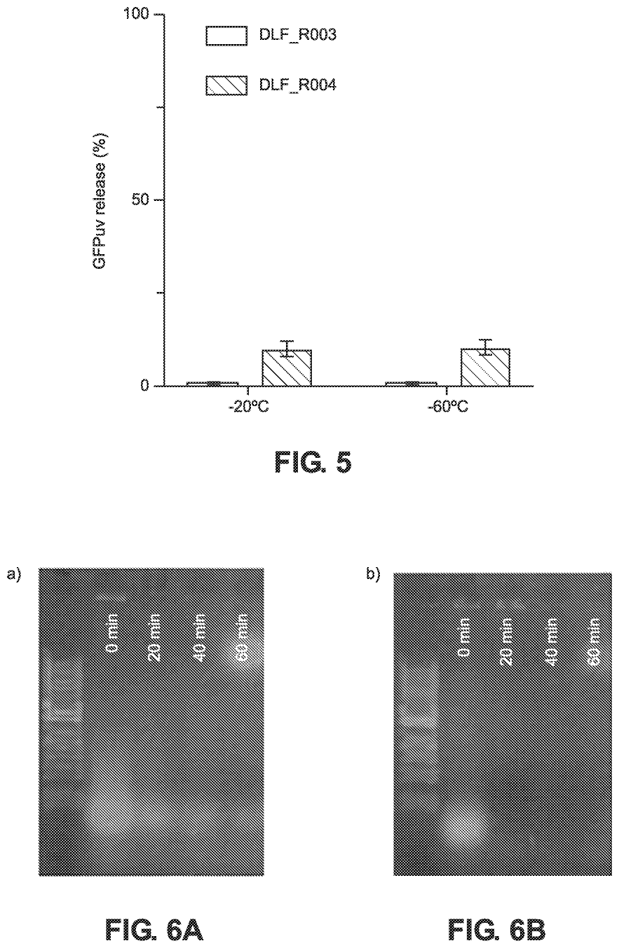

To further optimize protein release, the inventors evaluated the impact of a freeze-thaw cycle (FIG. 3B) on autolysis is demonstrated. Freeze-thaw is well known to provide cell wall and membrane disruption. As can be seen in FIG. 3B, a single 30-minute freeze-thaw after the addition of 0.1% Triton™-X100 at −20 degrees Celsius led to >90% release of GFP (FIG. 5).

### Example 3: Autohydrolysis

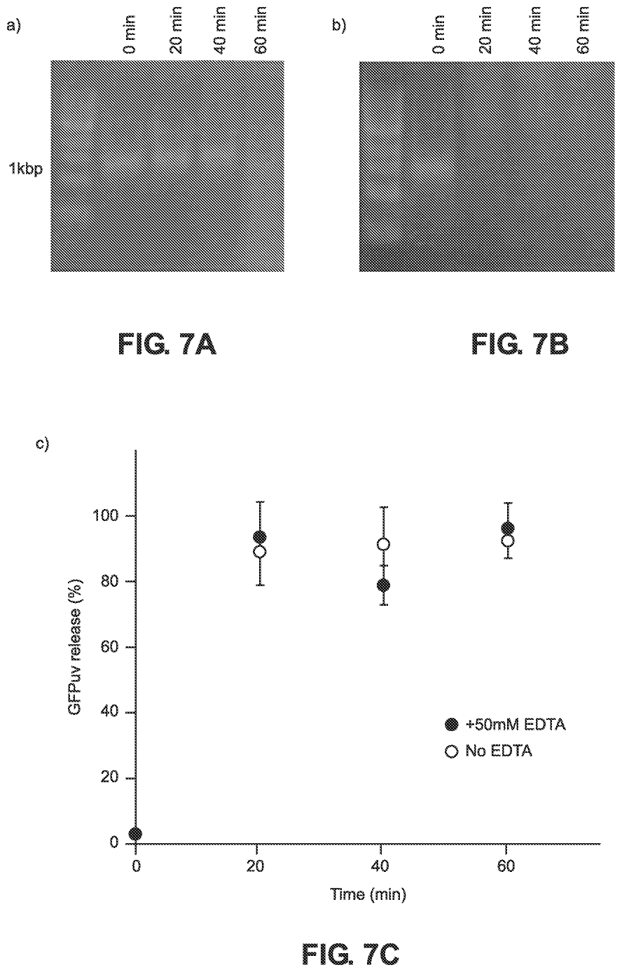

The inventors validated the autohydrolysis conferred by the benzonase to the autolysis/hydrolysis strain. To accomplish this, the inventors measured DNA/RNA hydrolysis as a function of time during cell autolysis. In the case of hydrolysis, cell lysates were more concentrated to be able to measure differences in DNA concentrations. Cell pellets were resuspended in 1/10th culture volume of 20 mM Tris buffer (pH=8.0), plus 2 mM MgCl₂. As a control, EDTA (50 mM) was optionally added prior to freeze thawed pellets to inhibit benzonase. Cell pellets were treated with 0.1% Triton™-X100 followed by a single 30-minute freeze thaw. After freeze thaw samples were incubated at 37° Celsius and samples taken to evaluate hydrolysis. 50 mM EDTA was added to samples to inhibit nuclease activity before analysis. Relative levels as well as the size of DNA/RNA were measured both by agarose gel electrophoresis. Results are given in FIG. 7.

Referring specifically to FIG. 7, autohydrolysis of DNA/RNA of strain DLF_R004 is detailed. A time course of DNA/RNA hydrolysis with (A) and without (B) EDTA (which inhibits benzonase by chelating Mg²⁺) is shown. C: A time course of autolysis and GFPuv release under autohydrolysis conditions using strain DLF_R004 bearing plasmid pHCKan-yibDp-GFPuv is shown.

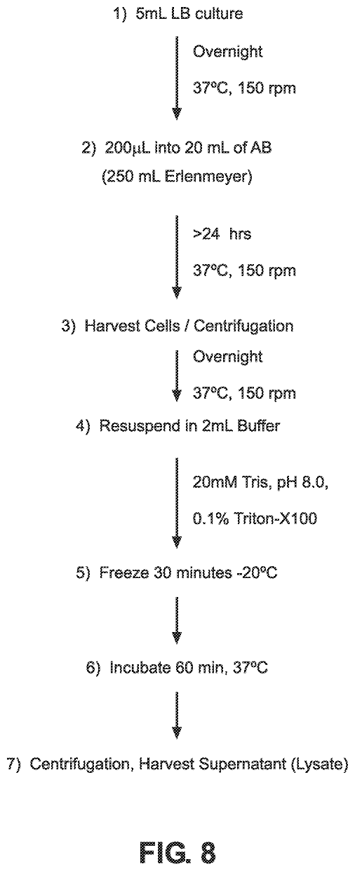

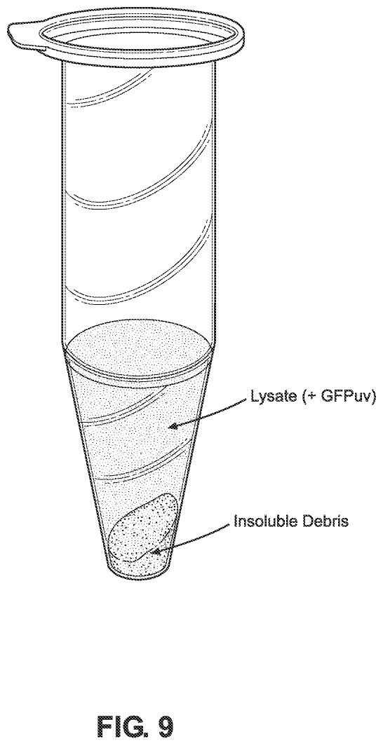

DNA hydrolysis, occurs in parallel with autolysis, and visible DNA/RNA was gone within 60 minutes of initiating autolysis. A protocol with more concentrated lysate was then evaluated for protein release using GFPuv, results of which are given in FIG. 7C and FIG. 6 for DNA analysis leading to a recommended routine expression and autolysis/hydrolysis protocol for shake flask cultures (outlined in FIG. 8). Refer to FIG. 9 for an example lysate generated using this protocol.

### Example 4: High Throughput Autolysis/Hydrolysis

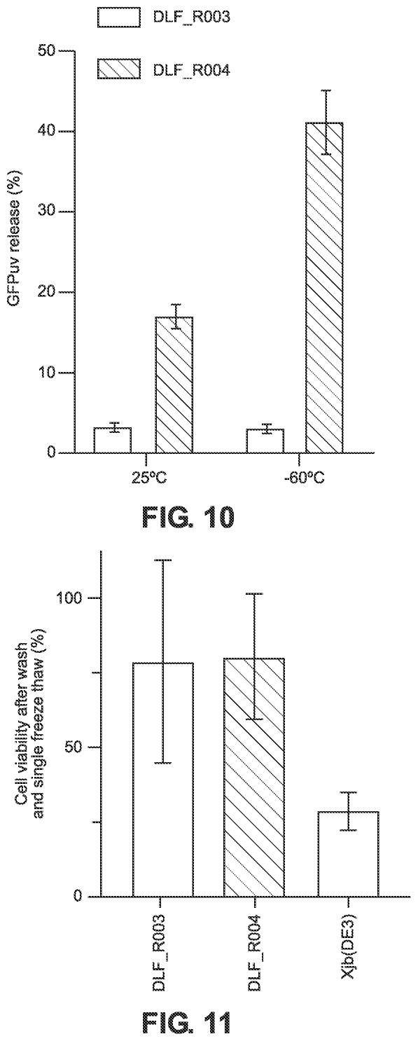

To build upon the successful autolysis and autohydrolysis observed in cells harvested from shake flask cultures, the inventors additionally validated this approach with high throughput microtiter-based expression. Autolysis/hydrolysis in microtiter plates greatly simplifies high throughput screening of proteins in crude lysates as well as proteins purified from crude lysates. As illustrated in FIG. 10, autolysis and protein release successfully scaled down to microtiter plates.

### Example 5. Stability of Uninduced Cells

A challenge with several current autolysis strains is sensitivity to free thaw during routine workflows, presumably due to leaky expression of the lysis proteins. And while DLF_R004 has demonstrated stability in autoinduction cultures, we confirmed that autolysis did not occur during routine freeze thaw cycles such as those used in preparing electrocompetent cells where not only are cells frozen and thawed but also thoroughly washed to remove ions including magnesium ions. The inventors tested the stability of electrocompetent cells for both DLF_R004 as well as another well known, readily available autolytic strain of E. coli, strain Xjb(DE3) from ZymoResearch. Xjb(DE3) relies on arabinose induction to induce lysozyme and autolytic behavior. In addition, the manufacturer recommends that excess magnesium is added to routine cultures to stabilize the cell wall of these cells, which is not feasible when preparing electrocompetent cells. Results of these competent cell studies are given in FIG. 11. While strain Xjb(DE3) suffered from unwanted lysis in these studies, DLF_R004, with tight control over expression of lysozyme and benzonase had increased stability during this process. Referring specifically to FIG. 11, the stability of uninduced strain DLF_R004 (an autolysis/hydrolysis strain), DLF_R003 (control), and autolysis strain E. coli Xjb were analyzed. Percent viability was measured after washing with ice-cold water twice, ice-cold 10% glycerol once and a single freeze thaw. Viability was measured as colony forming units after the freeze thaw normalized to colony forming unites before freeze thaw, multiplied by 100%.

### Conclusion

The Examples provided herein demonstrate the development of an improved strain of E. coli for not only autoinduction of protein expression but also of lysozyme and benzonase thereby enabling combined autolysis and auto DNA/RNA hydrolysis. This is the first combination of these two mechanisms to improve cellular lysis and DNA removal, and an example of the potential benefits of two stage production. This system enables>95% lysis and hydrolysis. Due to tightly controlled expression these strains are stable to shear forces in stirred tank bioreactors and even when subjected to freeze thaw cycles in deionized water, with 10% glycerol. Complete autolysis/hydrolysis as well as reduced lysate viscosity (due to oligonucleotide removal) allows for simplified liquid handling automation, useful in high throughput screening protocols. The mild detergents (0.1% Triton™-X100) used are also compatible with high throughout SDS-PAGE alternatives including capillary electrophoresis systems. In commercial production, the autoinduction of benzonase can remove the need to purchase nucleases for DNA removal and simplify purification and reduce costs.

Benzonase is difficult to inactivate and only denatures under conditions that most likely will impact the activity of any protein of interest. As a result, subsequent purification may be applied to remove benzonase. This is not an issue for routine shake flask expression or commercial scale production where additional downstream purification steps are expected. In applications for DNA/RNA modifying enzymes, addition purification can be administered. In sum, the method is well suited for routine shake flask expression and protein purification, as well as larger scale production. In addition, the approach further has applicability to the production of other intracellular products beyond proteins including polyhydroxyalkanoates (PHAs).

### Common Materials & Methods

Reagents and Media: Unless otherwise stated, all materials and reagents were of the highest grade possible and purchased from Sigma (St. Louis, Mo.). Luria Broth, lennox fonnulation with lower salt was used for routine strain and plasmid propagation and construction and is referred to as LB below. Working antibiotic concentrations were as follows: kanamycin (35 μg/mL) and apramycin (100 μg/mL). Auto induction Broth (AB) and FGM 10 media were prepared as previously reported.

Strains and Plasmids: Strain Xjb(DE3) was obtained from Zymo Research (Irvine, Calif.). E. coli strains DLF_R002 and DLF_R003 were constructed as previously reported. The autolysis/autohydrolysis strain: DLF_R004 was constructed using synthetic DNA. Briefly, Linear DNA (gBlock, IDT Coralville, Iowa) was obtained with the Lamba lysozyme and benzonase operon driven by a yibDp phosphate controlled promoter, preceded by a strong transcriptional tenninator and followed by an apramycin resistance marker (FIG. 1B). The nucA reading frame included its native N-terminal secretory signal (‘MRFNNKMLALAALLFAAQAS’ SEQ ID NO: 7). These sequences were flanked by homology arms targeting the deletion of the ompT protease. This cassette was directly integrated into the genome of strain DLF_R002 via standard recombineering methodology. The recombineering plasmid pSIM5 was a kind gift from Donald Court (NCI, https://redrecombineering.ncifcrf.gov/court-lab.html). OmpT deletion and autolysis/autohydrolysis operon integration was confirmed by PCR amplification and sequencing (Genewiz, N.C.). Plasmid pHCKan-yibDp-GFPuv (Addgene #127078) was constructed as previously reported.

Cell Growth & Expression: Shake flask cultures, BioLector™ studies microfennentations (microtiter plate cultivations) and 1 L instrumented fennentations were performed as described in Menacho-Melgar et al. Briefly, batch cultures utilized autoinduction broth (AB Media) and fermentations were performed using FGM IO media. Shake flask expression were performed at 150 rpm in baffled 250 mL Erlenmeyer flasks, with 20 mL of culture.

Lysis Measurements: DLF_R003 and DLF_R004 strains bearing plasmid pHCKan-yibDp-GFPuv were grown in LB overnight and later used to inoculate 250 mL shake flasks containing AB Media. After 24 hours, cells were harvested by centrifugation at 4000 rpm at 4° C. and resuspended in lysis buffer. Cultures were aliquoted in 1 mL samples. Lysis buffer consisted of either Buffer 1 or Buffer 2. Buffer 1 was used when hydrolysis was not needed and Buffer 2 for autohydrolysis. Buffer 1: phosphate buffer saline pH 7.4 (137 mM NaCl, 2.7 mM KCl, 8 mM Na2HPO4, and 2 mM KH2PO4) supplemented with 0.1% Triton™-X100 and Ix Halt Protease inhibitors (ThermoFisher Scientific, Waltham, Mass.). Buffer 2: 20 mM Tris, pH 8.0, 2 mM MgCl2 supplemented with 0.1% Triton™-X100 and Ix Halt Protease inhibitors. Cells were resuspended in 1/10 to ½ the original culture volume (in the case of MTPs). To lyse, cells were incubated in ice (0° C. experiments), preheated heat blocks (25 and 37° C. experiments) or prechilled tube racks (−20° C. and −60° C. experiments) for the indicated time. After lysis, samples were centrifuged at 4° C. at 13 000 rpm for one minute. Fluorescence readings were performed using a Tecan Infinite 200 plate reader in black 96 well plates (Greiner Bio-One, reference 655087) using 200 μl. Samples were excited at 412 nm (OmegaOptical, Part Number 3024970) and emission was read at 530 nm (OmegaOptical, Part Number 3032166) using a gain of 60. Fluorescence values were normalized to complete soluble protein release as obtained from sonicating one sample of each flask using a needle sonicator at 50% power output and 10 s/30 s on/off cycles for 20 minutes. Under these conditions, we found no more protein release with further sonication.

DNA Hydrolysis: DLF_R004 and DLF_R004 plus pHCKan-yibDp-GFPuv strains were grown overnight in LB. Overnight cultures were used to inoculate 20 mL of AB, in a 250 mL Erlenmeyer flask at 1% v/v in triplicate. Antibiotics were added as appropriate. Cultures were grown for 24 hours at 37° C. and 150 rpm. Cells were harvested by centrifugation and resuspended in 2 mL of Lysis/Hydrolysis Buffer (20 mM Tris, pH 8.0, 2 mM MgCl₂, 0.1% Triton™-X100, with or without 50 mM EDTA). After resuspension, cells were subjected to a single freeze thaw at −20° Celsius. Following freeze thaw samples were incubated at 37 degrees Celsius. Samples were taken every 20 minutes, and in the no EDTA reaction, EDTA was added to a final concentration of 50 mM. In the case of DLF_R004, samples were clarified by centrifugation and the supernatants analyzed via agarose gel electrophoresis. In the case of DLF_R004 plus pHCKan-yibDp-GFPuv, as GFPuv is also visualized under UV light used to visual agarose gels, after initial lysate clarification and supernatant sampling for GFPuv release, samples were heat denatured at 95° Celsius for 5 minutes, and then clarified again by centrifugation and the supernatants analyzed via agarose gel electrophoresis.

Microtiter Plate Expression and Autolysis: 96 well plate expression studies again utilized AB media according to Menacho-Melgar et al. using 100 μl of culture volume. After 24 hours of growth in AB, cells were harvested using a Vpsin plate centrifuge for 8 minutes at 3000 rpm. Supernatant was removed using a Biotek Plate washer/filler. 50 μl of Lysis Buffer (Buffer 1 above) was added, cells were resuspended by shaking, and placed at −60° C. for 30 minutes. After freezing cells were thawed for 10 minutes at 37° C., and lysates clarified by centrifugation again using a Vpsin plate centrifuge for 8 minutes at 3000 rpm. 5 μl lysate (supernatant) was collected and diluted 40-fold for analysis of GFPuv levels.

Strain Stability Measurements: DLF_R003, DLF_R004 and Xjb(DE3) strains were grown overnight in LB. Overnight cultures were used to inoculate 5 mL of LB at 2% v/v in triplicate. The new cultures were grown for 2-3 hours at 37° C. and 150 rpm until 0.6-0.8 OD600 was reached. At this point, samples were taken, diluted 250,000-fold and 50 μL were plated in LB agar plates. The rest of the cells were made electrocompetent by washing twice with 1 mL ice-cold water and once with 1 mL ice-cold glycerol. Cells were then frozen for 2 hours at −60° C. After thawing, samples were again diluted and plated as described above. Colonies were counted after incubating the agar plates overnight.

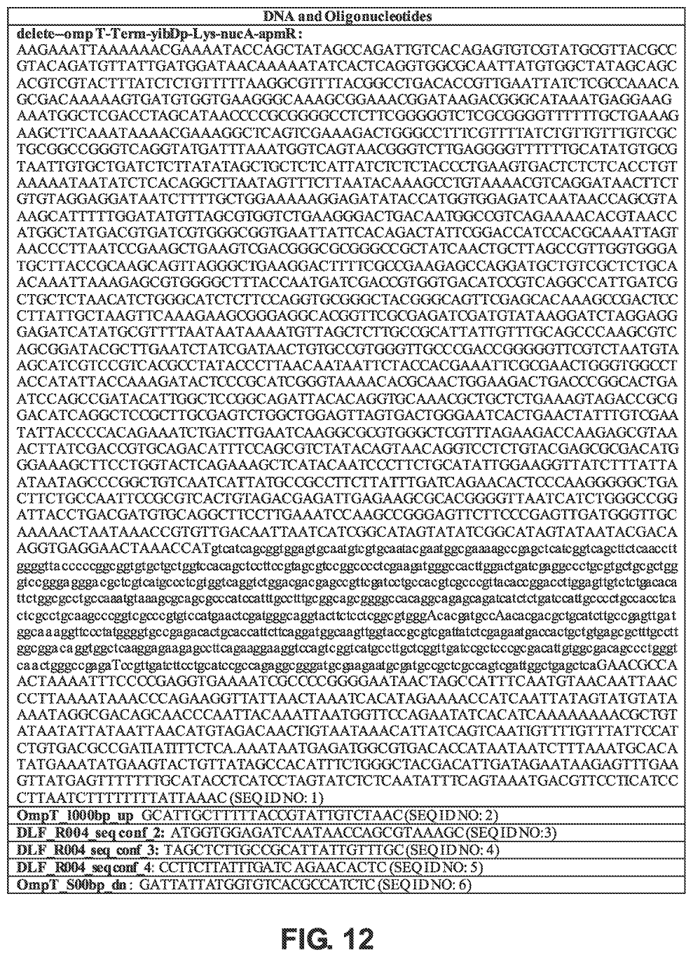

DNA and Oligonucleotides are found in FIG. 12.

One skilled in the art will readily appreciate that the present disclosure is well adapted to carry out the objects and obtain the ends and advantages mentioned, as well as those inherent therein. The present disclosure described herein are presently representative of preferred aspects, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. An engineered E. coli microorganism characterized by controlled autoinduction of cellular autolysis and DNA/RNA auto hydrolysis, the microorganism comprising one or more genes encoding a periplasmic lysozyme and one or more genes encoding a cytoplasmic nuclease, wherein genes encoding the periplasmic lysozyme and the cytoplasmic nuclease are operatively linked to a promoter that induces gene expression upon nutrient depletion from media containing the microorganism.

2. (canceled)

3. The engineered E. coli microorganism of claim 1, wherein the inducible promotor and the genes encoding a periplasmic lysozyme or the genes encoding a cytoplasmic nuclease are integrated as an operon in the chromosome of the microorganism.

4. The engineered E. coli microorganism of claim 1, wherein all genes encoding a periplasmic lysozyme or all genes encoding a cytoplasmic nuclease found within the microorganism are subject to expression by inducible promoter.

5. The engineered E. coli microorganism of claim 1, wherein the nutrient depleted from the media is inorganic phosphate.

6. The engineered E. coli microorganism of claim 1, wherein the periplasmic lysozyme is a lambda phage lysozyme.

7. The engineered E. coli microorganism of claim 1, wherein the periplasmic lysozyme is the Lambda R gene.

8. The engineered E. coli microorganism of claim 1, wherein the cytoplasmic nuclease is a benzonase.

9. The engineered E. coli microorganism of claim 1, wherein the cytoplasmic nuclease is the Serratia marcescens nucA gen.

10. The engineered E. coli microorganism of claim 1 further comprising a pathway for heterologous protein production wherein genes encoding enzymes essential for heterologous protein production are operatively linked to a promoter that induces gene expression upon nutrient depletion from media containing the microorganism.)

11. A method of cellular lysis and protein recovery comprising:
a) providing an engineered E. coli microorganism comprising one or more genes encoding a periplasmic lysozyme and one or more genes encoding a cytoplasmic nuclease, wherein genes encoding the periplasmic lysozyme and the cytoplasmic nuclease are expressed in the microorganism under the control of a promoter that induces gene expression upon nutrient depletion from media containing the microorganism in a growth phase;
b) growing the microorganism in a nutrient limited media;
c) inducing a microorganism stationary phase upon nutrient depletion, wherein the stationary phase is characterized by:
protein product expression, and
induction of the expression of the autolysis genes, wherein the autolysis enzymes are kept from inducing lysis until cell wall or membrane integrity is disrupted;
d) disrupting cell wall or membrane integrity;
e) collecting protein product.

12. The method of claim 11, wherein the protein product is heterologous and the engineered E. coli microorganism further comprised genes encoding enzymes essential for heterologous protein production operatively linked to a promoter that induces gene expression upon nutrient depletion from media containing the microorganism, and wherein the stationary phase is additionally characterized by induction of the expression of the heterologous genes.

13. The method of claim 11, wherein the engineered E. coli microorganism comprised both genes encoding a periplasmic lysozyme and genes encoding a cytoplasmic nuclease.

14. The method of claim 11, wherein, after inducing a stationary phase and prior to disrupting cell wall or membrane integrity, the cells are harvested by centrifugation.

15. The method of claim 11, wherein the step of disrupting cell wall or membrane integrity comprises at least one freeze thaw cycle, agitation, detergent addition or a combination thereof.

16. The method of claim 11, wherein the step of disrupting cell wall or membrane integrity comprises the addition of 0.1 weight % non-ionic detergent.

17. The method of claim 11, wherein step d) further comprises nucleotide hydrolysis by incubation at 37° C.

18. The method of claim 11, wherein the engineered E. coli microorganism comprises a lambda phage lysozyme or the engineered E. coli microorganism comprises a benzonase.

19. The method of claim 11, wherein the engineered E. coli microorganism comprises a Lambda R gene.

20. The method of claim 11, wherein the engineered E. coli microorganism comprises the Serratia marcescens nucA gene.


SEQUENCE LISTING (7 sequences)

SEQ ID NO: 1 - DNA, 288 bp, Artificial Sequence
  Synthetic
attataattaacatgtagacaacttgtaataaacattatcagtcaattgttttgtttattccatctgtgacgccgattattttctcaaaataatgagatggcgtgacaccataatctttaaatgcacatatgaaatatgaagtactgttatagccacatttctgggctacgacattgatagaataagagtttatgaagttatgagtttttttgcatacctcatcctagtatctctcaatatttcagtaaatgacgttccttcatcccttaatcttttttttattaaac

SEQ ID NO: 2 - DNA, 27 bp, Artificial Sequence
  Synthetic
gcattgctttttaccgtattgtctaac

SEQ ID NO: 3 - DNA, 29 bp, Artificial Sequence
  Synthetic
atggtggagatcaataaccagcgtaaagc

SEQ ID NO: 4 - DNA, 25 bp, Artificial Sequence
  Synthetic
tagctcttgccgcattattgtttgc

SEQ ID NO: 5 - DNA, 24 bp, Artificial Sequence
  Synthetic
ccttcttatttgatcagaacactc

SEQ ID NO: 6 - DNA, 24 bp, Artificial Sequence
  Synthetic
gattattatggtgtcacgcatctc

SEQ ID NO: 7 - protein, 20 aa, Artificial Sequence
  Synthetic
MRFNNKMLALAALLFAAQAS